(19) United States
(12) Patent Application Publication
(10) Pub. No.: US 2023/0256222 A1
(43) Pub. Date: Aug. 17, 2023

(54) DEVICE AND METHOD TO FACILITATE INSULIN INJECTION AND ABSORPTION

(71) Applicant: BULSAI, INC., Beverly Hills, MI (US)

(72) Inventors: William Joseph Ervin, Beverly Hills, MI (US); Matthew L. Miller, Grand Haven, MI (US); Mark E. Schafer, Ambler, PA (US)

(21) Appl. No.: 18/168,812

(22) Filed: Feb. 14, 2023

Related U.S. Application Data
(60) Provisional application No. 63/310,378, filed on Feb. 15, 2022.

(51) Int. Cl. A61M 37/00; A61B 5/00

(52) U.S. Cl. CPC A61M 37/0092; A61B 5/0095; A61M 2205/50; A61M 2205/581

(57) ABSTRACT

An illustrative example embodiment of a device that facilitates administering a fluid into an individual's body tissue includes an ultrasound transducer configured to emit sound waves and detect reflected sound waves. At least one processor is configured to: control operation of the ultrasound transducer in a first mode for detecting a condition of the individual's body tissue, determine a suitable administration site based on the transducer operating in the first mode, instigate an indication regarding the suitable administration site, and control operation of the ultrasound transducer in a second, different mode for enhancing absorption of the fluid at the suitable administration site.

### CROSS REFERENCE TO RELATED APPLICATION

This application claims priority to U.S. Provisional Application No. 63/310,378, filed Feb. 15, 2022.

### BACKGROUND

Many individuals with Type 1 diabetes have difficulty administering insulin manually and through the use of a pump because, over time, tissue changes such as scar tissue, lipohypertrophy, and cysts tend to develop between the epidermis and subcutaneous tissue where insulin is delivered. This is particularly true when an individual repeatedly administers insulin in the same location. Any of those conditions can cause minimal insulin to be absorbed into the body, causing higher than normal blood glucose levels. In some situations, an individual may administer a second dose at another site before the most recent dose has been fully absorbed, introducing a risk of too much insulin being absorbed.

### SUMMARY

An illustrative example embodiment of a device that facilitates administering a fluid into an individual's body tissue includes an ultrasound transducer configured to emit sound waves and detect reflected sound waves. At least one processor is configured to: control operation of the ultrasound transducer in a first mode for detecting a condition of the individual's body tissue, determine a suitable administration site based on the transducer operating in the first mode, instigate an indication regarding the suitable administration site, and control operation of the ultrasound transducer in a second, different mode for enhancing absorption of the fluid at the suitable administration site.

The various features and advantages of an example embodiment will become apparent to those skilled in the art from the following detailed description. The drawings that accompany the detailed description can be briefly described as follows.

### BRIEF DESCRIPTION OF THE DRAWINGS

FIG. 1 schematically illustrates a device for facilitating administering a fluid to an individual.

FIG. 2 shows an example configuration of a device having the features schematically shown in FIG. 1.

FIG. 3 is a flowchart diagram summarizing a method of operating a device having the features schematically shown in FIG. 1.

FIG. 4 schematically illustrates tissue layers at a suitable administration site.

FIG. 5 schematically illustrates tissue layers including an anomaly that may interfere with absorption of a fluid into the tissue.

FIG. 6 schematically illustrates selected features of a system including at least one device having the features schematically shown in FIG. 1.

### DETAILED DESCRIPTION

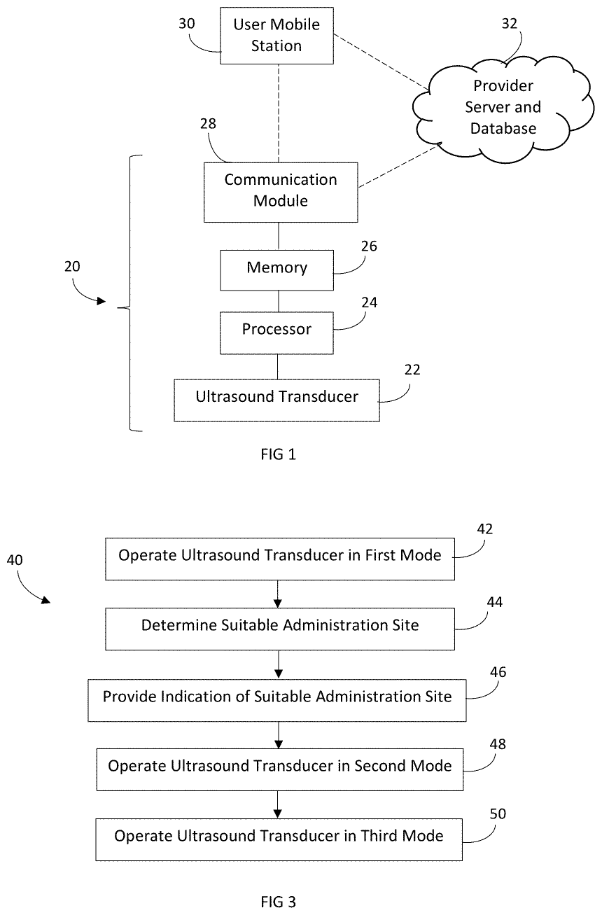

FIG. 1 schematically shows a device 20 that facilitates administering a fluid to an individual. In some embodiments, the device 20 is useful to assist in administering insulin to an individual who has diabetes. The following description refers to such an individual and insulin as the fluid administered to that individual. Embodiments of this invention are not necessarily limited to administering insulin or use by individuals who have diabetes. Other example fluids include hormones, blood thinners (Lovenox), and medications that stimulate blood cell production such as filgrastim (Neupogen).

The device 20 includes an ultrasound transducer 22 that is configured to emit ultrasound waves and to detect reflected waves. The ultrasound transducer 22 in some embodiments includes a single transducer. Other embodiments include an array of transducers. This description uses the term “ultrasound transducer” to refer generically to either type of embodiment unless the context indicates otherwise.

A computing device or processor 24 has associated memory 26. The processor 24 is configured, such as by being programmed, to control operation of the ultrasound transducer 22. In some embodiments, the processor 24 is configured to perform at least some analysis of an output from the ultrasound transducer 22.

The device 20 includes a communication module 28. In the illustrated example, the communication module 28 is configured for communications with a mobile station 30, which may belong to the user of the device 20, such as the individual who requires insulin. The mobile station 30 may be a smartphone, a tablet, or a notebook computer. The communication module 28 in some embodiments is configured for Bluetooth or other wireless communications with the mobile station 30

At least one of the communication module 28 and the user mobile station 30 is configured to communicate with a remote server 32, which may be part of a cloud computing system, for example. The communications between the remote server 32 and the device 20 may be direct or through the mobile station 30.

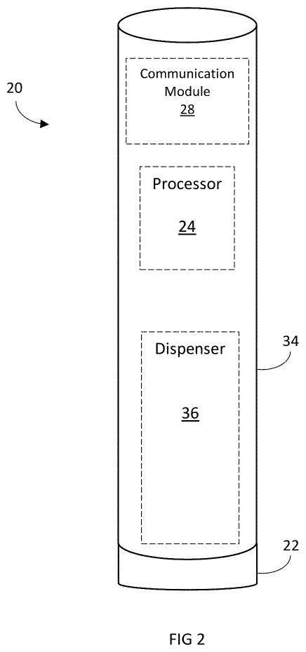

FIG. 2 schematically illustrates an example configuration of a device 20 having the features shown in FIG. 1. The example housing 34 has a generally cylindrical shape, such as a pen or wand, so that it can be held with one hand and manipulated into various positions by an individual. Other housing configurations are included in other embodiments. A dispenser 36 is appropriately associated or connected with the housing 34. For example, the housing 34 may be configured to receive a dispenser 36 at least partially within the housing 34. In other embodiments, the housing 34 has an exterior configured to be coupled with a dispenser in a manner that establishes a predetermined spatial relationship between the ultrasound transducer and the portion of the dispenser that directs insulin into the individual's body tissue. In some embodiments, the dispenser 36 comprises a hypodermic needle and a syringe. In other embodiments, the dispenser 36 is a needleless injector.

In some embodiments, the device 20 is entirely separate from a dispenser 36, such as an insulin pump or syringe.

FIG. 3 is a flowchart diagram 40 that summarizes an example method of using the device 20. At 42, the processor 24 controls the ultrasound transducer to operate in a first mode that is useful for finding a suitable site for injecting or otherwise administering insulin beneath the individual's skin. In the first mode, the ultrasound transducer 22 emits sound waves and detects reflected waves. When the ultrasound transducer 22 is placed against the individual's skin, the reflected waves provide information regarding a condition of the body tissue beneath the skin.

The ultrasound transducer 22 is configured to emit the sound waves through the individual's skin and to detect sound waves reflected from the individual's body tissue within a depth of no more than 20 mm beneath the individual's skin. That depth allows for detecting any anomalies within the tissue between the epidermis or skin layer and muscle tissue. Insulin injections are typically made into the subcutaneous fat layer just beneath the skin. Typical insulin needles are up to 14 mm long and the device 20 is focused on the tissue layers where insulin is introduced into the individual's body.

In an example embodiment, the ultrasound transducer 22 emits sound waves in a range from 5 to 20 MHz at an intensity in a range from 0.01 to 1 watt/cm² when operating in the first mode. This wavelength range and intensity range allow the device 20 to obtain information regarding the condition of the body tissue adjacent the transducer 22.

At 44, the processor 24 uses the output from the ultrasound transducer 22 to determine whether the body tissue at a current location of the ultrasound transducer 22 corresponds to a suitable administration site. In some embodiments, the processor 24 makes that determination independently. In other embodiments, the mobile station 30 is loaded with an application that allows the computing device of the mobile station 30 to make the determination and provide the determination to the processor 24.

If the body tissue at the location under consideration has the characteristics of a suitable administration site, the processor 24 causes the device 20 to provide an indication 46 to the user that injecting or otherwise administering insulin at that site is expected to result in desired absorption of the insulin. The indication may be visible, audible, haptic, or a combination of at least two of those.

According to the illustrated example, after the insulin has been introduced into the body tissue, the processor 24 controls the ultrasound transducer to operate in a second mode at 48, which facilitates or enhances absorption of the insulin. The ultrasound transducer emits sound waves in a range from 500 kHz to 10 MHz at an intensity in a range from 1 to 100 watt/cm² when operating in the second mode. This second mode of operation tends to induce a localized increase in tissue temperature, cavitation, or both. The second mode also provides some mechanical stimulation to the tissue at the administration site. Any of those or a combination of them promotes insulin absorption.

In some embodiments, the processor 24 controls the ultrasound transducer 22 to operate in the second mode for a predetermined length of time, such as one to ten minutes. The device 20 provides an indication to the user when the second mode begins and ends in some embodiments.

According to the illustrated example, at 50, the processor controls the ultrasound transducer to operate in a third mode that is useful for determining whether the introduced insulin has been absorbed. In an example embodiment, the ultrasound transducer 22 emits sound waves in a range from 5 to 20 MHz at an intensity in a range from 0.01 to 1 watt/cm² when operating in the third mode.

When first introduced, injected fluid forms an anechoic pool or bubble between layers of the tissue at the injection site. Such a pool or bubble is distinguishable from the surrounding tissue based on the output of the ultrasound transducer 22. As the fluid is absorbed by the body, the size of such a pool decreases, which is discernable based on the output of the ultrasound transducer 22. The third mode of operation includes a wavelength and intensity that is suitable for detecting the presence or absence of the fluid at the administration site.

In some embodiments, the insulin is injected while the ultrasound transducer 22 is operating in the first mode and the processor 24 determines that the insulin has been properly injected. The device 20 provides a successful injection indication in some such embodiments.

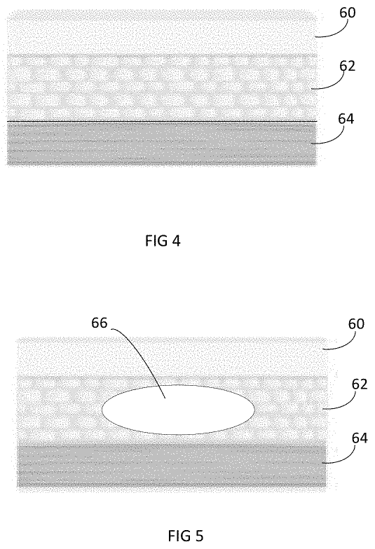

The device 20 implementing the method summarized in FIG. 3 provides information to an individual regarding the condition of body tissue at a potential administration site. FIG. 4 schematically shows a skin layer 60, subcutaneous fat layers 62, and muscle tissue 64. The subcutaneous fat layers 62 are generally uniform or consistent and that condition is detectable using the ultrasound transducer 22. When the layers of tissue appear consistent based on the output of the ultrasound transducer 22, the potential site is considered suitable because there are no anomalies, such as scarring, lipohypertrophy, or a cyst, that would interfere with desired insulin absorption.

FIG. 5 shows tissue layers that include an inconsistency 66, that corresponds to a cyst or localized lipohypertrophy, for example. That aberration in the tissue is likely to interfere with insulin absorption if the insulin were injected at that location. The device 20 notifies an individual of the presence of the anomaly in the tissue at such a potential injection site so the individual is able to avoid introducing insulin at that location. That indication can be any of visible, audible, haptic, or a combination of at least two of those indication types.

In some embodiments, the device 20 provides an indication regarding the condition of a potential administration site as an individual moves the ultrasound transducer 20 along a selected area of skin. For example, the indication changes from one that indicates an unfavorable site to one that indicates a suitable site depending on the corresponding outputs from the ultrasound transducer 22. The different indications help an individual locate a suitable injection site. In some embodiments, the device 20 communicates with the user's mobile station and a display or speaker of the mobile station provides the indication that guides the user to a suitable injection site.

In some embodiments, the device 20 is useful to provide information regarding an individual's body composition, such as a typical depth of fat tissue 62. That information is useful, for example, for recommending an injection needle size or an approach angle when injecting insulin. The information regarding body composition may also be used to calibrate the device 20 to ensure more accurate detection of anomalies that should be avoided.

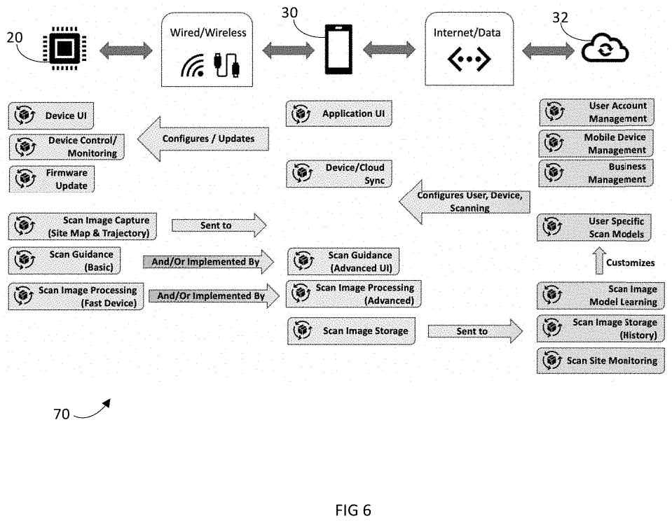

FIG. 6 schematically illustrates the functionality of a system 70 that includes the device 20, a user mobile station 30, and a remote server 32. The device 20 operates as described above, which is represented by the functions shown on the left side of the diagram. An application is loaded on the mobile station 30 that makes it capable of communicating with the device 20 and the server 32. The mobile station 30 has the ability to act as an intermediary between the server 32 and the device 20 in this example. The mobile station is also configured, through the application, to provide guidance to a user of the device 20 while scanning for a suitable administration site, to analyze the output of the ultrasound transducer 22 to determine whether a potential site is suitable for introducing insulin, to store data regarding tissue scan or injection absorption, and to provide any such information to the server 32.

The server 32 maintains a database of information regarding multiple users of devices 20, which may include user accounts, histories of detected anomalies, locations of detected anomalies, and other information that may be useful to a medical professional treating an individual with diabetes. The server 32 in some embodiments has computing devices that perform machine learning techniques for recognizing the tissue patterns or characteristics of an individual to calibrate or customize the device 20 to an individual's particular anatomy.

For example, the server 32 or the processor 24 develops a virtual ‘map’ of the patient that can be periodically or continuously updated. Tracking locations of sites to avoid because of a detected anomaly could be based on optical, magnetic, electromagnetic, or imaging technologies. When such a map is available, the device 20 may recommend a good location for the individual to begin scanning to find a suitable site for the next injection. Machine learning techniques are used in some such embodiments to learn where a suitable administration site is likely to exist. Additionally, the device 20 may be programmed so that after 60 days, for example, the device 20 recommends the individual go back to locations where anomalies were found and see if they are better. This way the device 20 could become customized to the user in terms of a body map showing good and bad locations, but also providing an indication when to revisit a ‘bad’ area. Some individuals may require 90 days before rechecking, for others maybe 30 days later that area is fine. Such information may be provided to the user through an interface on the user's mobile station 30, for example.

Embodiments of this invention facilitate administering a fluid, such as insulin, to an individual in a manner that promotes more consistent and reliable absorption of the fluid. Using a first diagnostic mode, a second therapeutic mode, and a third diagnostic mode enhances fluid absorption. Providing guidance to an individual regarding suitable sites for introducing the fluid, increasing absorption, and confirming absorption enhances the treatment and health of the individual.

The preceding description is exemplary rather than limiting in nature. Variations and modifications to the disclosed examples may become apparent to those skilled in the art that do not necessarily depart from the essence of this invention. The scope of legal protection given to this invention can only be determined by studying the following claims.

We claim:

1. A device that facilitates administering a fluid into an individual's body tissue, the device comprising:
an ultrasound transducer configured to emit sound waves and detect reflected sound waves; and
at least one processor configured to:
control operation of the ultrasound transducer in a first mode for detecting a condition of the individual's body tissue,
determine a suitable administration site based on the transducer operating in the first mode,
instigate an indication regarding the suitable administration site, and
control operation of the ultrasound transducer in a second, different mode for enhancing absorption of the fluid at the suitable administration site.

2. The device of claim 1, wherein the ultrasound transducer is configured to emit the sound waves through the individual's skin and to detect sound waves reflected from the individual's body tissue within a depth of no more than 20 mm beneath the individual's skin.

3. The device of claim 1, wherein
the ultrasound transducer emits sound waves in a range from 5 to 20 MHz when operating in the first mode, and
the ultrasound transducer emits sound waves in a range from 500 kHz to 10 MHz when operating in the second mode.

4. The device of claim 3, wherein
the ultrasound transducer emits the sound waves at an intensity in a range from 0.01 to 1 watt/cm² when operating in the first mode, and
the ultrasound transducer emits sound waves at an intensity in a range from 1 to 100 watt/cm² when operating in the second mode.

5. The device of claim 1, wherein the at least one processor is configured to
control operation of the ultrasound transducer in a third mode for detecting a change in an amount of the fluid in the individual's body tissue, and
instigate an indication regarding absorption of the fluid based on the ultrasound transducer operating in the third mode.

6. The device of claim 5, wherein
the ultrasound transducer emits sound waves in a range from 5 to 20 MHz when operating in the first mode,
the ultrasound transducer emits sound waves in a range from 500 kHz to 10 MHz when operating in the second mode, and
the ultrasound transducer emits sound waves in a range from 5 to 20 MHz when operating in the third mode.

7. The device of claim 1, comprising an injector configured to inject the fluid into the individual's body tissue and wherein the ultrasound transducer is situated adjacent the injector.

8. The device of claim 7, comprising an indicator configured to provide at least one of a visible indication, an audible indication or a haptic indication when the injector is situated to inject the fluid into the suitable administration site.

9. The device of claim 1, wherein
the processor is configured to determine whether a portion the individual's body tissue is the suitable administration site by determining whether the portion of the individual's body tissue includes an interruption to a predetermined tissue pattern.

10. The device of claim 9, wherein
the fluid comprises insulin, and
the interruption to the predetermined tissue pattern is based on at least one of scar tissue, lipohypertrophy, or a cyst.

11. A method of facilitating administration of a fluid into an individual's body tissue, the method comprising:
operating an ultrasound transducer in a first mode;
detecting a condition of the individual's body tissue based on operating the ultrasound transducer in the first mode;
determining a suitable administration site based on the detected condition;
providing an indication regarding the suitable administration site;
operating the ultrasound transducer in a second, different mode for enhancing absorption of the fluid at the suitable administration site.

12. The method of claim 11, wherein the ultrasound transducer is configured to emit the sound waves through the individual's skin and to detect sound waves reflected from the individual's body tissue within a depth of no more than 20 mm beneath the individual's skin.

13. The method of claim 11, wherein
operating the ultrasound transducer in the first mode comprises emitting sound waves in a range from 5 to 20 MHz, and
operating the ultrasound transducer in the second mode comprises emitting sound waves in a range from 500 kHz to 10 MHz.

14. The method of claim 13, wherein
operating the ultrasound transducer in the first mode comprises emitting the sound waves at an intensity in a range from 0.01 to 1 watt/cm², and
operating the ultrasound transducer in the second mode comprises emitting the sound waves at an intensity in a range from 1 to 100 watt/cm².

15. The method of claim 11, comprising
operating the ultrasound transducer in a third mode for detecting a change in an amount of the fluid in the individual's body tissue, and
providing an indication regarding detected absorption of the fluid.

16. The method of claim 15, wherein
operating the ultrasound transducer in the first mode comprises emitting sound waves in a range from 5 to 20 MHz,
operating the ultrasound transducer in the second mode comprises emitting sound waves in a range from 500 kHz to 10 MHz, and
operating the ultrasound transducer in the third mode comprises emitting sound waves in a range from 5 to 20 MHz.

17. The method of claim 11, comprising injecting the fluid into the individual's body tissue using an injector situated adjacent the ultrasound transducer.

18. The method of claim 11, wherein providing the indication of the suitable injection site comprises providing at least one of a visible indication, an audible indication or a haptic indication.

19. The method of claim 11, wherein determining the suitable administration site comprises determining whether the portion of the individual's body tissue includes an interruption to a predetermined tissue pattern.

20. The method of claim 19, wherein
the fluid comprises insulin, and
the interruption to the predetermined tissue pattern is based on at least one of scar tissue, lipohypertrophy, or a cyst.
